(19) United States
(12) Patent Application Publication
(10) Pub. No.: US 2008/0269568 A1
(43) Pub. Date: Oct. 30, 2008

(54) METHODS FOR VALIDATING A RADIOTHERAPY PLAN BY A DOSIMETRY SERVICE

(75) Inventors: David F. Lewis, Monroe, CT (US); Lawrence E. Reinstein, Lake George, NY (US)

Correspondence Address: INTERNATIONAL SPECIALTY PRODUCTS;WILLIAM J. DAVIS, ESQ., 1361 ALPS ROAD, WAYNE, NJ, 07470, US

(73) Assignee: ISP INVESTMENTS INC., Wilmington, DE (US)

(21) Appl. No.: 11/739,873

(22) Filed: Apr. 25, 2007

(51) Int. Cl. A61B 5/00

(52) U.S. Cl. 600/300

(57) ABSTRACT

Described are various methods for validating a patient's radiotherapy plan. One possible method comprises receiving a dose distribution at a dosimetry service, receiving dose data at the dosimetry service from a radiation detection device at a treatment site removed from the dosimetry service, the data having been gathered in accordance with the dose distribution, and evaluating the dose data in comparison to the dose distribution to prepare a validation report.

### BACKGROUND

The present invention relates generally to a dosimetry service for application in radiotherapy.

Film dosimetry for radiotherapy requires expensive equipment for data analysis and highly trained technicians to calibrate and operate the equipment. Traditionally, film dosimetry has been done using silver halide films. The response of silver halide films is energy dependent and they are light sensitive. Furthermore, because of the challenges of maintaining consistent post-exposure development the dose-density response of a silver halide film is difficult to calibrate. Hospitals and clinics that administer radiotherapy also administer many other procedures, so there is limited opportunity for the medical physicist or dosimetrist to develop radiotherapy film dosimetry expertise.

Accordingly, there is a need for a dosimetry service that uses a film that is energy independent, not light sensitive, does not require post-exposure development or processing, and is more easily calibrated. Additionally, there is a need for such a service that is able to operate more cost effectively. The dosimetry service may be more cost effective because it provides the most skilled professionals using the most technically advanced measurement techniques and equipment that is properly calibrated.

### SUMMARY OF THE INVENTION

In one aspect, the present invention provides a method for validating a radiotherapy plan using a dosimetry service. The method comprises receiving a dose distribution at a dosimetry service, receiving dose data at the dosimetry service from a radiation detection device at a treatment site removed from the dosimetry service, the data having been gathered in accordance with the dose distribution, and evaluating the dose data in comparison to the dose distribution to prepare a validation report.

In another aspect, the present invention provides another possible method for validating a patient's radiotherapy plan using a dosimetry service. The method comprises: receiving recorded radiation information at a dosimetry service, receiving a dose distribution at the dosimetry service, measuring the recorded radiation information, and comparing the measured recorded radiation information to the dose distribution for preparation of a validation report. Additional steps may be included, such as sending a film configured to record radiation information from the dosimetry service to a treatment site removed from the dosimetry service, loading the film into a phantom by the dosimetry service before sending the film to the treatment site, providing the treatment site with instructions for exposing the film, and/or sending a validation report from the dosimetry service to the treatment site or the person requesting the validation.

In yet another aspect, the present invention provides another possible method for validating a patient's radiotherapy plan using a dosimetry service. Here, the method comprises: receiving recorded radiation information at a dosimetry service, the recorded radiation information contained on a film comprising a radiation sensitive material, evaluating the recorded radiation information on the film to convert the recorded radiation information into measurement data, and sending the measurement data to a customer analysis site removed from the dosimetry service for validation of a dose distribution.

Other aspects of the present invention will become apparent from the following description, the accompanying drawings and the appended claims.

### BRIEF DESCRIPTION OF THE DRAWINGS

FIG. 1 is a flow diagram of one embodiment of a method for validating a radiotherapy plan where a dosimetry service receives dose data;

FIG. 2 is a flow diagram of another embodiment of a method for validating a radiotherapy plan where a film dosimetry sends films to a treatment site;

FIG. 3 is a flow diagram of another embodiment of a method for validating a radiotherapy plan using a dosimetry service; and

FIG. 4 is a contour plot showing lines of equal dose over a plane through the patient's treatment site.

### DETAILED DESCRIPTION

The following detailed description is intended to be representative only and not limiting as to the possible methods for validating a radiotherapy plan using a dosimetry service. Many variations can be devised by one skilled in this area of technology, which are included within the scope of the present invention. The following detailed discussion of the various alternative and preferred embodiments will illustrate the general principles of the invention, examples of which are illustrated in the accompanying drawings.

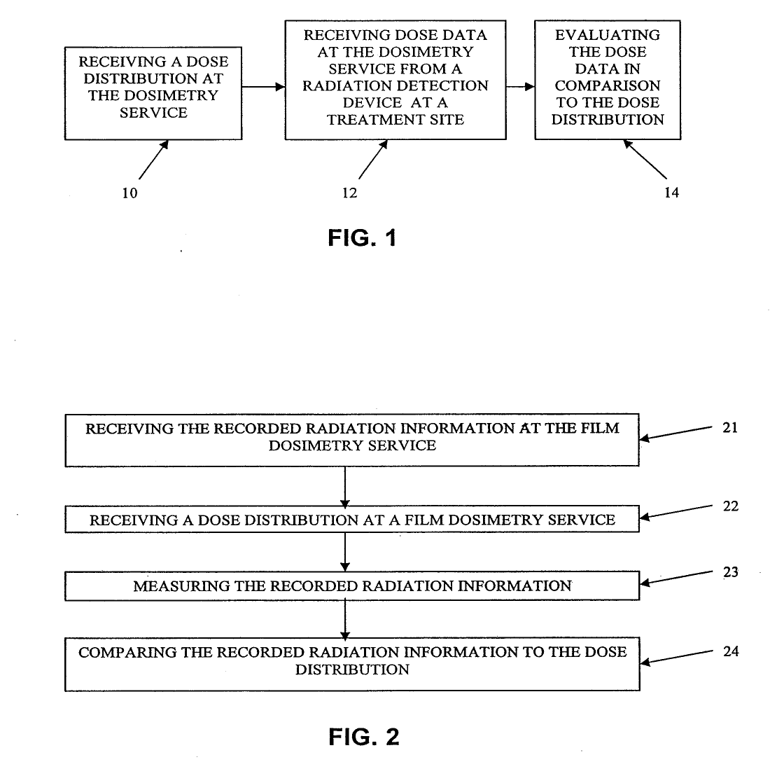

As illustrated in FIG. 1, one embodiment of the present invention is a method for validating a radiotherapy plan using a dosimetry service. Radiotherapy is the medical use of ionizing radiation, such as X-rays, gamma rays, β-rays, electron beams, proton beams and other photon and particle beams, to treat disease. The most common disease treated by radiotherapy is cancer. Radiotherapy may also be used to treat non-malignant conditions, such as, but not limited, to treatment of severe thyroid eye disease, pterygium, and prevention of keloid scar growth. Radiotherapy may be external radiotherapy or internal radiotherapy used for curative, adjuvant, palliative, or radical treatment.

The embodiment of the method in FIG. 1 validates a patient's radiotherapy plan by having a dosimetry service receive a dose distribution 10, receive dose data from a radiation detection device at a treatment site removed from the dosimetry service 12, and evaluate the dose data in comparison to the dose distribution 14. In another embodiment, the method includes an additional step of sending a validation report to the customer who requested the validation or to the treatment site. The dose distribution may be received by the dosimetry service from a person requesting the validation, the site that will perform the irradiation, or from a clinician. The receiving of the dose distribution and dose data 10, 12 may be by any mode that will get the information to the dosimetry service. Likewise, the sending of the validation report may be by any mode that will get the information to the person requesting the validation. The modes of travel may be, but are not limited to, mail, FedEx® carrier or the like, personal delivery or pickup, email or other electronic form or delivery, or any combination thereof. In one embodiment, the dose data is sent to the dosimetry service electronically.

In one embodiment, the radiation detection device may be an electronic portal imaging device. Electronic portal imaging devices include but are not limited to ion chamber arrays, diode arrays, and amorphous silicon arrays. In another embodiment the radiation detection device may be a film comprising radiation-sensitive material, which is described below in more detail. The film may be a radiochromic film. In one embodiment the radiochromic film may be a film made using lithium pentacosa-10,12-diynoate filamentary particle technology. In one embodiment, the dose data is received electronically by the dosimetry service.

The radiation detection device may be at a treatment site removed from the dosimetry service. The term “treatment site removed from the dosimetry service” as used herein means where one group of people are responsible for exposing the chosen medium to radiation, and a separate group of people at another location, even within the same building, company, or division of a company, are responsible for receiving the medium or data contained in or on the medium and performing the measurement and analysis involved in the evaluation.

The patient's radiotherapy plan may be any treatment plan using radiation that is proposed by a qualified medical worker for the treatment of the patient's disorder, disease, tumor, or the like. Qualified medical workers include clinicians, medical physicists and dosimetrists. In one embodiment, the dosimetry service receives the radiotherapy plan, but in other embodiments the dosimetry service receives a dose distribution calculated from the radiotherapy plan. A dose distribution is the result of the treatment plan being applied to a phantom containing the film, or other film test conditions. In one embodiment, the dose distribution can be based on a single radiation beam, while in other embodiments the dose distribution can be based on the sum of all radiation beams in the treatment plan. In another embodiment, the radiotherapy plan may be a test radiotherapy plan, i.e., one not created for a particular patient. The radiotherapy plan may be, but is not limited to, an intensity modulated radiation therapy plan, a stereotacetic radiosurgery plan, a 3-dimensional conformal radiotherapy plan, or an image guided radiation therapy, fractionated stereotacetic radiotherapy, respiratory gated treatment or helical tomotherapy.

Intensity modulated radiation therapy (“IMRT”) is a technology that allows a clinician to implement a highly conformal, even non-convex, dose distribution plan. IMRT is a dose plan and treatment delivery that is optimized using inverse or forward planning techniques for modulated beam delivery, using either a binary collimator, or with conventional MLC system using either “sliding window” or “step and shoot” modes. IMRT may employ a dynamic multileaf collimation to shape not only the profile of the beam, but also to vary the intensity of the beam over its area. The IMRT plan also includes dose planning objectives and constraints, criteria for target and critical structure expansions, 3-D dose distributions, and dose volume histogram analysis for targets and critical structures. IMRT makes it possible to apply more conformal dose distributions and to increase the daily treatment fraction to the target volume while decreasing the dose delivered to normal tissue.

Stereotacetic radiosurgery is also called radiation surgery, radiosurgery, stereotacetic external-beam radiation, stereotacetic radiation therapy, and stereotaxic radiosurgery. Stereotacetic radiosurgery is a radiation therapy procedure that uses special equipment to position the patient and to deliver with precision a dose of radiation, usually in a one-day session, to the treatment site and not to normal tissue. This procedure does not use invasive surgery. It is used to focus radiation beams and deliver them to the treatment site to treat abnormalities, tumors, functional disorders, or the like. Stereotacetic Radiosurgery may also be “fractioned,” such that the patient receives the radiation over a period of weeks.

As used herein, “dose data” means any form a recorded radiation information gathered by a radiation detection device when testing the radiation to be administered for the dose distribution of a radiotherapy plan.

The validation report may be a written, oral, or electronic report explaining the correlation between the dose distribution(s) in a patient's radiotherapy treatment plan and the recorded radiation information found on the film. The sending of the validation report may be by any of the modes of travel above, in addition to any electronic transmission or media, such as email, CD, or any electronic storage device, or any combination thereof.

FIG. 2 illustrates another embodiment of a method to validate a patient's radiotherapy plan. A dosimetry service receives recorded radiation information from a treatment site for evaluation 21. The recorded radiation information may have been generated by any method known in the art of radiotherapy. In one embodiment, the recorded radiation may have been generated by irradiating a film configured to record radiation information at the treatment site. The dosimetry service also receives a dose distribution 22 that corresponds to the recorded radiation information. Then the dosimetry service measures the recorded radiation information 23 and evaluates the measured recorded radiation information in comparison to the dose distribution for preparation of a validation report 24. The validation report can explain the correlation between the radiotherapy plan's dose distribution and the recorded radiation information on the films. Additionally, the dosimetry service may send a film comprising a radiation sensitive material to the treatment site. The film may comprise a radiation sensitive material and may be configured to record radiation information.

The radiation sensitive film may contain any type of radiation sensitive active component. This includes silver based active components and radiochromic components. In one embodiment, the film is a radiochromic film. Radiochromic film is a self-developing film that changes color as a result of exposure to ionizing radiation. Radiochromic films are available from International Specialty Products under its GAFCHROMIC® product line, which includes but is not limited to GAFCHROMIC® MD-55, GAFCHROMIC® MD-22, GAFCHROMIC® HD-810, GAFCHROMIC® HS, GAFCHROMIC® HS-14, GAFCHROMIC® RTQA-1010, GAFCHROMIC® RTQA-1010P, GAFCHROMIC® RTQA-111, GAFCHROMIC® EBT, GAFCHROMIC® XR Type R, GAFCHROMIC® XR-CT, GAFCHROMIC® XR-QA films.

The GAFCHROMIC® radiochromic film's active ingredient is a microcrystalline, radiation sensitive monomer that is dispersed in a gelatin matrix and coated onto a polyester film base. When the active monomeric component is exposed to ionizing radiation, a polymerization reaction is initiated, resulting in the production of a dye polymer. Since the polymer is by nature, a dye, the exposure produces coloration within the film. The amount of polymer produced, and by extension the depth of the color change, is proportional to the amount of energy absorbed in the active layer.

In another embodiment, the radiochromic film may be made using lithium pentacosa-10,12-diynoate (LiPCDA) filamentary particle technology. A method for producing and using a lithium salt of a polyacetylene as radiation sensitive filaments is described in United States Patent Application Publications No. US 2006/0134551 and No. US 2004/0197700, and in Provisional U.S. Patent Application Ser. No. 60/459,559, all of which are incorporated herein by reference in their entirety. In accordance with this invention a radiation sensitive lithium salt of a C₆ to C₆₄ conjugated polymerizable polyacetylene having at least one terminal carboxylic acid or carboxylate group is provided in the form of hair-like or bristle-like filaments and size measured in microns (μm) wherein the length to width ratio of said filaments is at least 5:1 and as high as 5000:1 or more up to several thousand to one.

The film comprising radiation sensitive material may be a plurality of films. In one embodiment, the plurality of films has at least one of each of a control film, a calibration film, and a test film. In another embodiment, the plurality of films contains multiple test films corresponding to each radiation field the customer would like to have evaluated from the patient's radiotherapy treatment plan. In yet another embodiment, the plurality of films may have a plurality of control films, calibration films, and test films as needed to evaluate the patient's radiotherapy treatment plan. As the complexity of the patient's radiotherapy treatment plan increases the number of films needed in the plurality of films is likely to increase, and the dosimetry service can supply all the films necessary to meet the customer's needs.

Some, or all, of the plurality of films comprising the radiation sensitive material may be provided by the dosimetry service pre-loaded into one or more phantoms, which are sent to the treatment site. A phantom is a device to simulate the patient's anatomy and place the film or films into an environment approximating the environment at the patient's treatment site. The films are placed into the phantoms to simulate the effect of the patient's anatomy on the ionizing radiation to be used for the patient's radiotherapy treatment plan. Phantoms are commonly constructed from tissue-equivalent or water-equivalent plastics. A phantom may be anatomically shaped, or formed from geometrically shaped solid plastic slabs, or it may be comprised of a set of solid blocks appropriately shaped for the purpose.

The control film is not to be radiated because it acts as a background test to determine if the plurality of films was exposed to a condition, other than the customer's radiation exposure, that may have affected the condition of other films in the package. If the irradiation trials stretch over several days, a control film per day may be necessary. Even if the irradiation trials are separated by a significant amount of time on the same day a different control film may be necessary. The significant amount of time is to be determined by the technician, clinician, or scientist running the irradiation trials.

The calibration film may be irradiated with a known radiation field, which may correspond to a dose to be administered in the patient's radiotherapy treatment plan or to selected values that are necessary to calibrate the machine being used over the necessary range of radiation to be administered in the patient's radiotherapy treatment plan. The calibration film may be irradiated with a plurality of known radiation fields, or with a combination of doses of known radiation fields.

The test film may be irradiated with a known radiation field in accordance with the patient's radiotherapy treatment plan. In one embodiment, the test film is irradiated with a radiation field that corresponds to a dose of radiation to be administered as part of the patient's radiotherapy treatment plan. In another embodiment, the test film may be irradiated with a plurality of or combination of known radiation fields.

A control film may be placed into a phantom and not irradiated. The calibration film and test film may be separately placed into phantoms and irradiated with the known radiation field or fields that correspond to radiation fields from the patient's radiotherapy treatment plan. The phantom is then placed into the known radiation field and the radiation is projected onto the film. After irradiation, the film comprising radiation sensitive material configured to record radiation information may contain recorded radiation information. The recorded radiation information may take any of the forms explained above.

The dosimetry service may prepare a package of radiation sensitive films for the treatment site. A package of films contains at least one film for each patient radiation field, at least one film for calibration and at least one control film. Each film in the package is separately labeled, and in one embodiment each label is unique. Optionally, the films may be scanned or otherwise measured prior to dispatch to the customer. In the package, the films are protected from harmful environmental exposure—e.g. light, moisture, heat, radiation, etc. Optionally some, or all, of the radiation sensitive films may be loaded into phantoms as described above.

When the dosimetry service receives the film comprising radiation sensitive material or a plurality thereof, the film or plurality of film may include a control film that has not been irradiated, a calibration film having been irradiated with one or more known radiation fields, and a test film having been irradiated with a known radiation field in accordance with the patient's radiotherapy treatment plan. There may be a plurality of any of the control film, calibration film, or test film. The control film, calibration film, and test film may contain recorded radiation information from being irradiated.

The dosimetry service receives the recorded radiation information from the treatment site for evaluation of the recorded radiation information contained on the film or plurality of film thereof. The recorded radiation information received may be a latent image or an image in the form of a color change, optical density pattern, a change in reflection, transmission, refractive index, or the like, depending upon the type of film used. In one embodiment using radiochromic film, the radiochromic film's recorded radiation information would be visible as a color change, which becomes progressively darker in proportion to the dose of radiation absorbed. The radiation sensitive film contains the recorded radiation information because the film was irradiated while at the treatment site. The irradiation may be administered in accordance with the dosages the customer wants to have evaluated.

The dosimetry service also needs to receive a patient's radiotherapy treatment plan. The receiving of the patient's radiotherapy treatment plan may be by any mode that will get the plan from the customer, medical worker, or treatment site to the dosimetry service. The modes of travel may be, but are not limited to, mail, FedEx® carrier or the like, personal delivery or pickup, email or other electronic form, or any combination thereof.

The treatment plan comprises a description of all or some of the following beam parameters for each radiation beam to be used in the treatment of a patient: collimator jaw positions, source to surface distance (SSD), gantry angle, collimator angle, customized block design, multi-leaf collimator (MLC) leaf positions, and dynamic MLC leaf files. The treatment plan generally contains information pertaining to the calculated spatial distribution of radiation doses to be applied to a patient in one or more treatment sessions. During any treatment session the patient may be treated by exposure to one of more fields of radiation. The radiation fields are projected onto the treatment site from one or more orientations. Fields may be applied singly, or in sequence. Alternatively the shape and intensity of the treatment field may be continuously varied. In addition, the orientation of the radiation beam with respect to the patient may change. The beam may be stepped between orientations, i.e. the beam is turned off, moved to a new orientation and then turned on, or the orientation of the beam may change continuously. Information contained in the treatment plan relates to the spatial distribution of radiation doses exposed on the radiation detection device.

Dose distribution relates to a spatial distribution of doses applied to the radiation detection device. The dose distribution may be a spatial distribution in one, or more dimensions as allowed by the capability of the radiation detection device. The radiation detection device may respond to radiation at a point, along a line, over a planar area, over a curved surface, or over a 3-dimensional volume.

Then the dosimetry service measures the recorded radiation information found on the film received at the dosimetry service, and evaluates the measurements in comparison to the patient's radiotherapy treatment plan. The measuring may be by any method, machine, or apparatus that is capable of reading the recorded radiation information depending upon the type of radiation sensitive film being used. In one embodiment where radiochromic film was used the color change is measured and quantified as an absorbed dose of radiation. The color change may be measured with any densitometer, scanner, spectrophotometer, or other equipment that can detect the color change. In one embodiment, the color change on the radiochromic film is measured using a film scanner, which creates a film scan image. The plurality of films received by the dosimetry service contains one or more calibration films where areas of the calibration films have been exposed to one or more known doses of radiation. Measurement of the recorded radiation information on the calibration film provides a correlation between the measured response of the film and the radiation dose. This correlation provides the means whereby measured images of the patient films can be converted to dose information. The evaluation step may be completed by comparing the dose information to the corresponding radiation field from the patient's radiotherapy treatment plan. The evaluation of the doses may be performed by specialized computer software that compares the dose information measured on the films to the dose distribution contained within the treatment plan.

In one embodiment the dose information measured on a radiation sensitive device such as a radiation sensitive film represents a 2-dimensional distribution of radiation doses. Evaluation of a patient treatment plan involves a comparison of at least one measured distribution of doses with the distribution of doses exposed onto that film as calculated from the treatment plan. The evaluation could be facilitated in a variety of ways such as by printing maps of measured and calculated dose distributions and overlaying such maps for the purpose of comparison. More frequently the evaluation is performed with the use of computer software by means of which a variety of qualitative and quantitative comparisons are made. An example is the software FilmQA™ Validation from 3Cognition LLC. This software calculates and displays 2-dimensional contour maps of a measured dose-distribution overlaid on the distribution calculated from the treatment plan. The software provides the capacity to compare profiles of dose distribution in any selected orientation. It also employs quantitative evaluation tools typically used by Medical Physicists to assess patient radiotherapy treatment plans such as the calculation of the % dose-difference between measurement and plan as well as assessment of the gamma quality index and distance-to-agreement functions. The last two functions are discussed by W. B. Harms, et al. in a publication in Medical Physics Vol. 25(10) p 1830-36. These functions are commonly used by Medical Physicists to set quantitative criteria for accepting or rejecting a IMRT patient treatment plans.

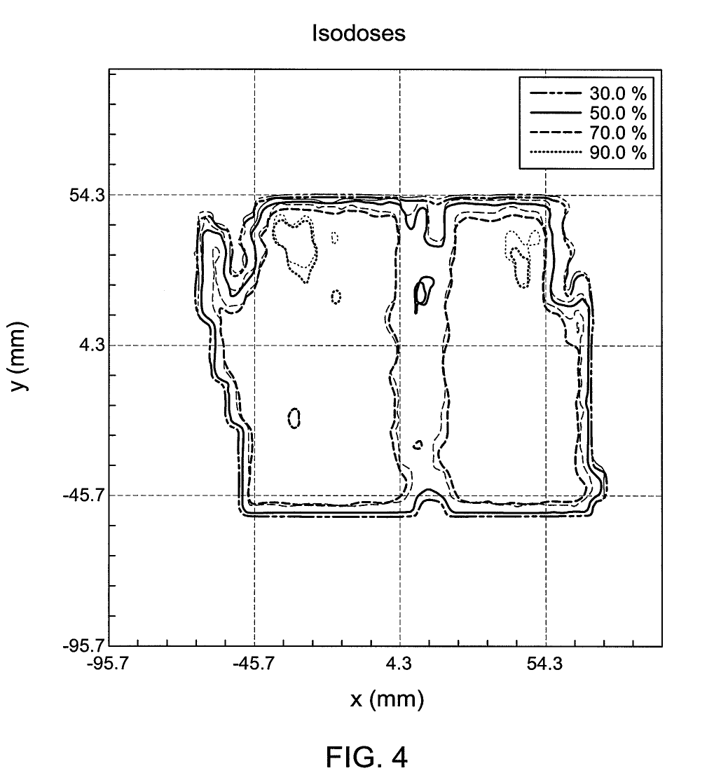

One example of evaluating the dose distribution is presented as a contour plot in FIG. 4. This Figure is a contour plot showing lines of equal dose over a plane through the patient's treatment site. The contour plot shows a series of isodose lines measured in that plane using a radiation sensitive film. The isodose lines showing the measured values are superimposed over a matching series of isodose lines calculated from the patient treatment plan. An isodose line traces the positions of points in the measurement plane having equal dose. The isodoses shown in FIG. 4 are based as a % of the maximum radiation dose planned to be administered in the plane. In the example, the contour lines represent doses of 180 cGy for the 90% isodose line, 140 cGy for the 70% isodose line, 100 cGy for the 50% isodose line and 60 cGy for the 30% isodose line. In an ideal case the isodose contours for the measurement would be perfectly matched with the isodose lines from the plan. In practice there are differences. A Medical Physicist well trained in the art of IMRT dosimetry is able to examine the correspondence of the isodose lines and decide whether the treatment plan is suitable for administration or must be modified before it is given to the patient.

Another embodiment of the method above may also include sending to the customer or the treatment site instructions for exposing the film comprising a radiation sensitive material and a validation report with the results of the evaluation of the patient's radiotherapy treatment plan. In another embodiment, the instructions may additionally have information on how to properly handle the films, and how to use and/or load a phantom, and how to get the irradiated films back to the dosimetry service. The sending of the instructions may be by any of the modes of travel described above, or combinations thereof. In one embodiment, the instructions direct the customer or treatment site to place the film or films into a phantom for irradiation. The phantoms may have been supplied by the dosimetry service along with the films, but un-loaded. In another embodiment the customer or treatment site may provide their own phantoms.

Another embodiment of a method for validating a radiotherapy plan using a dosimetry service has the irradiation performed by the dosimetry service. For this embodiment, the dosimetry service receives a patient's radiotherapy treatment plan and instructions from a customer, irradiates a film comprising a radiation sensitive material in accordance with the customer's instructions, evaluates the recorded radiation information recorded on the film, and sends the customer a validation report.

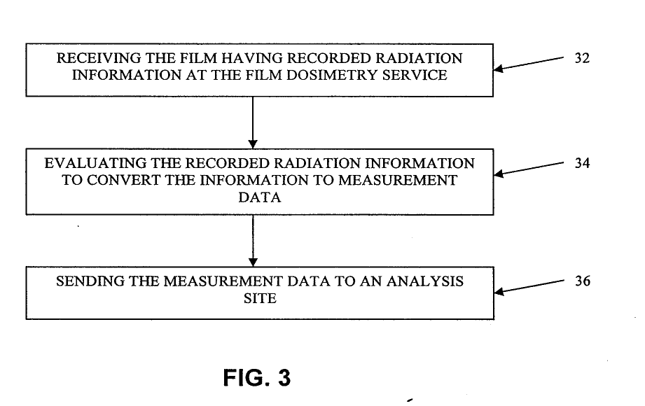

Another embodiment, as shown in FIG. 3, is a method for validating a radiotherapy plan using a dosimetry service where the dosimetry service receives the film comprising radiation sensitive material having recorded radiation information thereon 32, evaluates the recorded radiation information on the film to convert the recorded radiation information to measurement data 34; and sends the measurement data to a customer analysis site 36 for validation of a dose distribution. In one embodiment, the dosimetry service may send a film comprising a radiation sensitive material to a treatment site.

The film comprising radiation sensitive material and the recorded radiation information may be sent by any of the ways described above. In one embodiment the dosimetry service sends the film pre-loaded into a phantom to the treatment site. In another embodiment, the film may be a plurality of films as described above. The plurality of films may be packaged into phantoms before being sent to the treatment site.

After receiving the film containing recorded radiation information, the dosimetry service evaluates the recorded radiation information 34. The evaluation may be done by any of the methods, machines, or apparatus as explained above. As shown in FIG. 3, the dosimetry service is to convert the recorded radiation information found on the film to measurement data 34. The measurement data may be recorded such that the recorded radiation information is correlated with the dosage of radiation administered, which is the dosage the patient may receive in their treatment.

The dosimetry service then sends the measurement data to a customer analysis site 36. The customer analysis site may be the same site as the treatment site or a site removed from the treatment site, such as a hospital, clinic, or an expert in such analysis. The customer analysis site removed from the film dosimetry service may be located similarly in relation to the dosimetry service as described above for a treatment site that is removed from the dosimetry service.

Although the present invention is shown and described with respect to certain aspects, it is obvious that various modifications will become apparent to those skilled in the art upon reading and understanding the specification and the appended claims. The present invention includes all such improvements and modifications and is limited only by the scope of the claims.

What is claimed:

1. A method for validating a radiotherapy plan, the method comprising:
receiving a dose distribution at a dosimetry service;
receiving dose data at the dosimetry service from a radiation detection device at a treatment site removed from the dosimetry service, the data having been gathered in accordance with the dose distribution; and
evaluating the dose data in comparison to the dose distribution to prepare a validation report.

2. The method of claim 1 further comprising:
sending a validation report from the dosimetry service to the person requesting the validation.

3. The method of claim 1 wherein the radiation detection device is an electronic portal imaging device or radiation-sensitive film.

4. The method of claim 3 wherein the electronic portal imaging device is at least one of an ion chamber array, a diode array, and an amorphous silicon array.

5. The method of claim 3 wherein the radiation-sensitive film is radiochromic film.

6. The method of claim 1 wherein the dose data is received electronically.

7. A method for validating a radiotherapy plan, the method comprising the steps of:
receiving recorded radiation information at a dosimetry service,
receiving a dose distribution at the dosimetry service;
measuring the recorded radiation information; and
comparing the measured recorded radiation information to the dose distribution for preparation of a validation report.

8. The method of claim 7 further comprising:
sending a film configured to record radiation information from the dosimetry service to a treatment site removed from the dosimetry service.

9. The method of claim 8 further comprising:
loading the film into a phantom by the dosimetry service before sending the film to the treatment site.

10. The method of claim 8 further comprising:
providing the treatment site with instructions for exposing the film; and
sending a validation report from the dosimetry service to the treatment site or the person requesting the validation.

11. The method of claim 10 wherein the instructions direct the treatment site to place the film into a phantom for irradiation.

12. The method of claim 8 wherein the film is radiochromic film.

13. The method of claim 12 wherein the radiochromic film is made using lithium pentacosa-10,12-diynoate filamentary particle technology.

14. The method of claim 12 wherein the film comprises at least one control film, at least one calibration film, and at least one test film corresponding to each radiation field to be evaluated as part of the dose distribution.

15. The method of claim 14 wherein the films are packaged by the dosimetry service to protect the films from harmful environmental exposure.

16. The method of claim 7 wherein the dose distribution is from an intensity modulated radiation therapy treatment plan or a stereotacetic radiosurgery plan.

17. The method of claim 7 wherein measuring the recorded information comprises scanning the film to create a film scan image and converting the film scan image to a dose measurement.

18. The method of claim 7 wherein receiving the recorded radiation information is as an electronic version or file.

19. A method for validating a radiotherapy plan, the method comprising:
receiving recorded radiation information at a dosimetry service, the recorded radiation information contained on a film comprising a radiation sensitive material;
evaluating the recorded radiation information on the film to convert the recorded radiation information into measurement data; and
sending the measurement data to a customer analysis site removed from the dosimetry service for validation of a dose distribution.

20. The method of claim 19 further comprising:
sending the film from the dosimetry service to a treatment site removed from the dosimetry service, the film configured to record radiation information.

21. The method of claim 20 wherein the film is radiochromic film.

22. The method of claim 21 wherein the radiochromic film is made using lithium pentacosa-10,12-diynoate filamentary particle technology.

23. The method of claim 19 wherein the film is a plurality of radiation sensitive films comprising a control film, a calibration film, and a test film that corresponds to each dose distribution.

24. The method of claim 20 further comprising:
providing the customer with instructions for handling and exposing the film.

25. The method of claim 19 wherein evaluating the recorded information comprises scanning the film to create a film scan image, and converting the film scan image to a dose measurement.
